(19) United States
(12) Patent Application Publication
(10) Pub. No.: US 2020/0383700 A1
(43) Pub. Date: Dec. 10, 2020

(54) INSTRUMENT PORT

(71) Applicant: COVIDIEN LP, Mansfield, MA (US)

(72) Inventors: JACOB C. BARIL, NORWALK, CT (US); MATTHEW A. DININO, NEWINGTON, CT (US); GEORGE S. MATTA, PLAINVILLE, MA (US); NICOLETTE R. LAPIERRE, WINDSOR LOCKS, CT (US); SAUMYA BANERJEE, HAMDEN, CT (US); ROY J. PILLETERE, NORTH HAVEN, CT (US); JUSTIN J. THOMAS, NEW HAVEN, CT (US)

(21) Appl. No.: 16/431,809

(22) Filed: Jun. 5, 2019

(51) Int. Cl. A61B 17/34

(52) U.S. Cl. CPC A61B 17/3423; A61B 17/3462; A61B 2017/00862; A61B 2017/345; A61B 17/3498

(57) ABSTRACT

An instrument port for surgical instruments includes a patient interface body configured to engage a patient access point, e.g., wound. The patient interface body includes a distal end configured to facilitate insertion into the patient access point and a proximal end including a lip defining a cuff therein. The patient interface body including a channel defined therethrough that extend from proximal to distal ends. A flexible seal is configured to securely seat within the cuff and includes an instrument opening configured to align with the channel. A seal guard is configured to sit atop the flexible seal and includes an instrument opening configured to align with the channel and the flexible seal instrument opening. A port cover is configured to engage the lip to encapsulate the flexible seal and the seal guard and defines an opening configured to align with the channel, the flexible seal instrument opening and the seal guard opening.

### BACKGROUND

### Technical Field

The present disclosure relates to instrument ports for surgical instruments and, more particularly, to an instrument port for surgical instruments with a seal guard configured to protect the integrity of instrument port.

### Background of Related Art

Surgical techniques and instruments have been developed that allow the surgeon to perform an increasing range of surgical procedures with minimal incisions into the skin and body tissue of the patient. Minimally-invasive surgery has become widely accepted in many medical specialties, often replacing traditional open surgery. Unlike open surgery, which requires a long incision, minimally-invasive procedures, such as endoscopy or laparoscopy, are performed through one or more short incisions, with much less trauma to the body.

In laparascopic and endoscopic surgical procedures, a small “keyhole” Incision or puncture is made in a patient's body, e.g., in the abdomen, to provide an entry point for a surgical access port which is inserted into the incision and facilitates the insertion of specialized instruments used in performing surgical procedures within an internal surgical site. In other surgeries, the port may be utilized to provide access to a containment bag or specimen bag.

Minimally-invasive surgical procedures are performed throughout the body and generally rely on obtaining access to an internal surgical site through a relatively small patient access point, often less than one centimeter in diameter, to the surgical site. One method of providing such a pathway is by inserting a cannula and trocar assembly through the skin of the patient. Commonly, to place the trocar-cannula, the penetrating tip of the obturator of the trocar is pushed through the skin and underlying tissue until the distal end of the cannula is within the body cavity. Alternatively, some port devices have a blunt obturator for placing the cannula through a previously-made incision, wound or, in some cases, a mechanical ring (disposed on an outer periphery of a specimen bag). Once the port has been properly positioned, the port is then available as a pathway between the surgical site and the exterior of the patient's body through which the surgeon may introduce the various surgical instruments required to perform the desired procedures. Surgical instruments insertable through a port include forceps, clamps, scissors, shavers, morcellators, probes, flexible or rigid scopes, staplers and cutting instruments.

In some procedures, a wall of a body cavity is raised by pressurization of the body cavity to provide sufficient working space at the surgical worksite and/or to allow a trocar to penetrate the body cavity or a port to be inserted through a wound or incision. The process of distending the abdomen wall from the organs enclosed in the abdominal cavity is referred to as insufflation, During a laparascopic procedure (endoscopy in the abdominal cavity), insufflation is achieved by introducing an insufflation gas, such as carbon dioxide, nitrogen, nitrous oxide, helium, argon, or the like, through a Veress needle or other conduit inserted through the abdominal wall, to enlarge the area surrounding the target surgical site to create a larger, more accessible work area called a pneumoperitoneum. The surgeon is then able to perform the procedure within the body cavity by manipulating the instruments that have been extended through the surgical access devices. The manipulation of such instruments within the internal body is limited by both spatial constraints and the need to maintain the body cavity in an insufflated state.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

A surgical instrument provided in accordance with aspects of the present disclosure includes a patient interface body configured to engage a patient access point, e.g., wound or mechanical ring affixed to another port or specimen bag. The patient interface body includes a distal end configured to facilitate insertion into the patient access point and a proximal end including a lip defining a cuff therein. The patient interface body includes a channel defined therethrough. A flexible seal is included and is configured to securely seat within the cuff. The flexible seal includes an instrument opening defined therein configured to align in registration with the channel.

A seal guard is included and is configured to sit atop the flexible seal. The seal guard includes an instrument opening defined therein configured to align in registration with the channel and the flexible seal instrument opening. A port cover is included and is configured to securely engage the lip to encapsulate the flexible seal and the seal guard. The port cover defines an opening configured to align in registration with the channel, the flexible seal instrument opening and the seal guard opening.

In aspects according to the present disclosure, the patient access point is a wound, mechanical instrument ring or natural orifice of a patient and the patient interface body is configured to mechanically engage the wound, mechanical instrument ring or natural orifice.

In aspects according to the present disclosure, the flexible seal is made from silicone. In other aspects, the seal guard is made from a material that is harder than the flexible seal. In still other aspects, the seal guard is made from a material that is more lubricous than the flexible seal.

In aspects according to the present disclosure, the opening defined in the flexible seal is smaller than the opening defined in the port cover. In other aspects, the opening defined in the seal guard is smaller than the opening defined in the flexible seal. In still other aspects, the flexible seal and the seal guard each include a series of additional openings defined therein configured to operably engage a corresponding plurality of bosses disposed within the cuff.

In aspects according to the present disclosure, the port cover is secured to the lip via screws that operably engage the lip to the port cover. In other aspects, the port cover is secured to the lip via a plurality of screws that operably engage the corresponding plurality of bosses disposed within the cuff.

A surgical instrument provided in accordance with other aspects of the present disclosure includes a patient interface body configured to engage a patient access point, e.g., wound or mechanical ring affixed to another port or specimen bag. The patient interface body includes a distal end configured to facilitate insertion into the patient access point and a proximal end including a lip defining a cuff therein. The patient interface body includes a channel defined therethrough extending from the proximal end to the distal end.

A flexible seal is included and is configured to securely seat within the cuff. The flexible seal includes an instrument opening defined therein having a first diameter and which is configured to align in concentric registration with the channel. A seal guard is included and is configured to sit atop the flexible seal. The seal guard includes an instrument opening defined herein having a second diameter smaller than the first diameter and which is configured to align in concentric registration with the channel and the flexible seal instrument opening.

A port cover is included and is configured to securely engage the lip to encapsulate the flexible seal and the seal guard. The port cover defines an opening having a third diameter slightly larger than the first diameter and which is configured to align in concentric registration with the channel, the flexible seal instrument opening and the seal guard opening.

In aspects according to the present disclosure, the patient access point is a wound, mechanical instrument ring or natural orifice of a patient and the patient interface body is configured to mechanically engage the wound, mechanical instrument ring or natural orifice.

In aspects according to the present disclosure, the flexible seal is made from silicone. In other aspects, the seal guard is made from a material that is harder than the flexible seal. In still other aspects, the seal guard is made from a material that is more lubricous than the flexible seal.

In aspects according to the present disclosure, the flexible seal and the seal guard each include a series of additional openings defined therein configured to operably engage a corresponding plurality of bosses disposed within the cuff.

In aspects according to the present disclosure, the port cover is secured to the lip via screws that operably engage the lip to the port cover. In other aspects, the port cover is secured to the lip via a plurality of screws that operably engage the corresponding plurality of bosses disposed within the cuff.

In aspects, the patient interface body includes helical threads to facilitate insertion of the instrument port within the patient access point.

### BRIEF DESCRIPTION OF THE DRAWINGS

Various aspects and features of the present disclosure described herein with reference to the drawings wherein:

FIG. 1 is a perspective view of an instrument port provided in accordance with the present disclosure including a patient interface body and a port cover;

FIG. 2 is an exploded view of the instrument port of FIG. 1 showing the port cover, seal guard, flexible seal and patent interface aligned for assembly;

FIG. 3 is a top view of the instrument port of FIG. 1; and

FIG. 4 is a side, cross sectional view taken along line 4-4 of FIG. 1.

### DETAILED DESCRIPTION

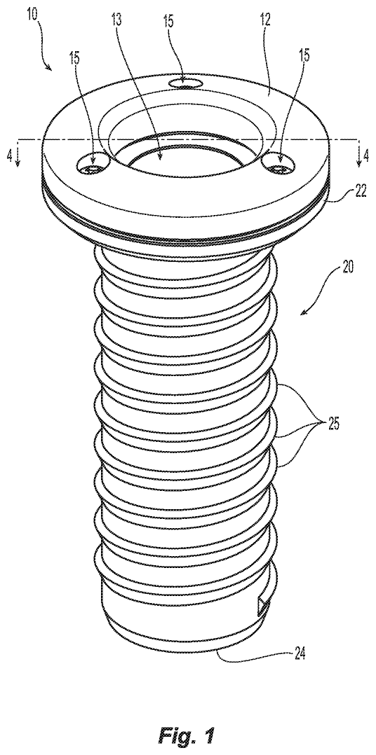

Referring generally to FIG. 1, an instrument port 10 is shown and includes a patient interface body 20 configured to engage a patient access point (e.g., wound or incision within the patient, mechanical ring attached to a collection bag or other portion of a collection bag, or a natural patient orifice) and a port cover 12 configured to support an instrument (not shown) therethrough. Patient interface body 20 includes a lip 22 defined at a proximal end thereof and a tip 24 disposed at a distal end thereof. A helical threading 25 is disposed about an outer peripheral surface of the patient interface body 20 and extends from the proximal lip 22 to the distal tip 24. An instrument channel 27 is defined through patient interface body 20 and extends from the proximal lip 22 to the distal tip 24. Instrument channel 27 is configured to allow insertion of an instrument up to 25 mm in diameter. Other channel sizes 27 are envisioned. The distal tip 24 may include a reduced diameter or reliefed portion to facilitate entry into the patient access point (e.g., wound or a mechanical ring (not shown)).

Helical threading 25 is configured to engage the inner peripheral surface of the patient access point and secure the instrument port 10 to the patient. For example, threading 25 may be configured to enable rotational advancement and engagement of instrument port 10 within the patient access point, or may be configured to engage a corresponding mechanical structure (not shown) disposed within the wound such as, for example, a mechanical ring disposed within another port or enclosure (e.g., specimen bag (not shown)). Threading 25 may be made from a flexible material (e.g., silicone or the like) that easily deforms to engage irregular aspects of a wound or mechanical ring to maintain integrity of the pneumoperitoneum during a surgical procedure. The patient interface body 20 may be entirely made from a semi-rigid material for similar purposes.

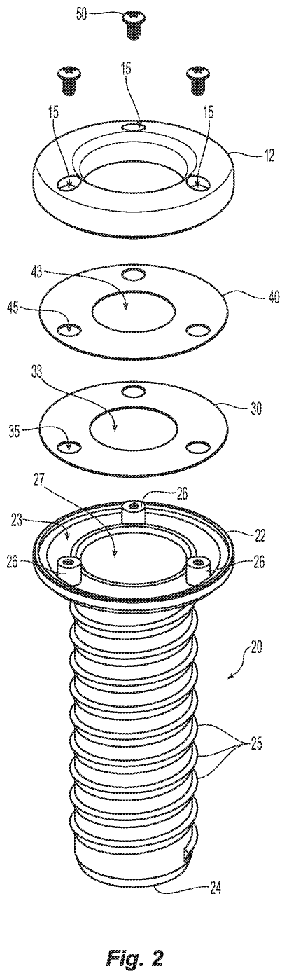

As shown in FIG. 2, port cover 12 is configured to operably engage the proximal lip 22 via one or more screws 50 or in a snap-fit fashion. Proximal lip 22 may include one or more corresponding bosses 26 that align to engage screws 50 during assembly. Port cover 12 may also be ultrasonically welded atop lip 22 as an alternative to screws 50 or snap-fit engagement. Port cover 12 also includes a central opening 13 defined therein configured to permit insertion of an instrument therethrough and into instrument channel 27.

Port cover 12 is configured to encapsulate a flexible seal 30 and a seal guard 40. Flexible seal 30 is configured to sit within a recessed cuff 23 defined within proximal lip 22. As series of additional openings 35 are defined through flexible seal 30 that align with the corresponding bosses 26 of proximal lip 22 and securely seat flexible seal 30 within the cuff 23 of proximal lip 22. Flexible seal 30 includes an instrument opening 33 defined therein configured to receive an instrument (not shown) therethrough.

Flexible seal 30 is configured to tightly seal around the outer periphery of the shaft of the instrument to protect the integrity of the pneumoperitoneum while allowing the instrument to freely slide therethrough and move therearound as needed during surgery. Flexible seal 30 may be made from any known flexible material that accomplishes this purpose, e.g., silicone. Instrument opening 33 of flexible seal 30 may be dimensioned slightly smaller than opening 13 such that the shaft of the instrument remains properly sealed during introduction and subsequent positioning.

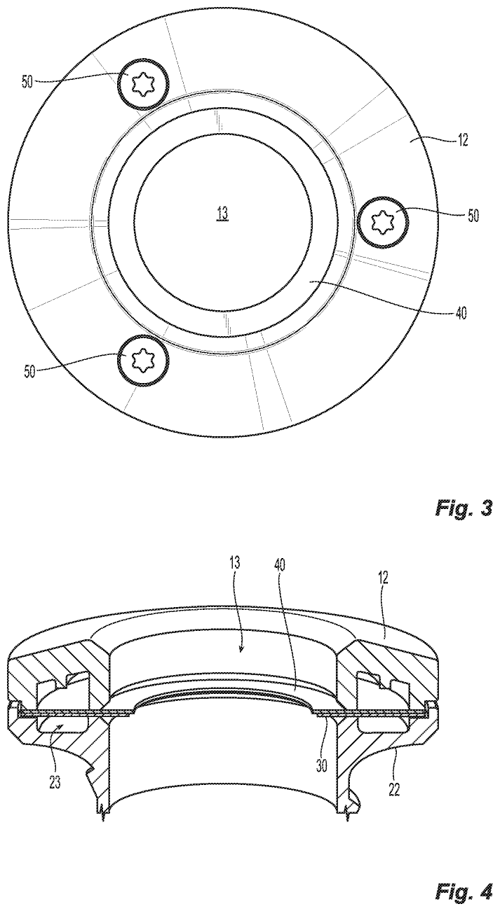

Seal guard 40 is configured to sit atop flexible seal 30 within recessed cuff 23 defined within proximal lip 22. As series of additional openings 45 are defined through seal guard 40 that align with the corresponding bosses 26 of proximal lip 22 to securely seat seal guard 40 within the cuff 23 of proximal lip 22. Seal guard 40 includes an instrument opening 43 defined therein configured to receive the instrument (not shown) therethrough. Seal guard 40 is typically made from a harder, more lubricious material than flexible seal 30 and is configured to prevent the flexible seal 30 from being ripped or torn during normal usage. As such and as shown in FIG. 3, the instrument opening 43 of seal guard 40 is typically dimensioned slightly smaller than the instrument opening 33 of flexible seal 30.

FIG. 4 shows a cross section detailing one particular arrangement of the instrument port 10 including the proximal lip 22 of the patient interface body 20 housing the flexible seal 30 and seal guard 40 within the cuff 23 and the port cover 12 encapsulating the same secured by the screws 50 (See FIG. 3). Reversing the order of assembly of the flexible seal 30 and seal guard 40 may work for one or more particular types of instruments or instrument shafts. Moreover, and as mentioned above, the port cover 12 may be operably coupled to the patient interface body 20 by other known methods of attachment including, snap-fit, ultrasonic welding, overmold, screw-fit, etc.

Once assembled, the instrument port 10 is inserted into a patient access point (e.g., wound or through a mechanical ring in a specimen bag (not shown)). The instrument port 10 may be simply pushed into engagement with the patient access point or twisted and pushed into the patient access point to engage. The patient interface body 20 is configured to maintain the integrity of the pneumoperitoneum. Once the instrument port 10 is inserted in situ, an instrument or instrument shaft (not shown) may be introduced through port opening 13, opening 43 of the seal guard, opening 33 of the flexible seal, and through the instrument channel 27 and into the pneumoperitoneum. The flexible seal 30 (and the seal guard 40) cooperate to encapsulate the instrument and/or instrument shaft in a fluid tight manner to insure the integrity of the pneumoperitoneum during insertion and instrument manipulation. The seal guard 40 ensures that the flexible seal 30 does not tear or rip during use.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An instrument port for surgical instruments, comprising:
a patient interface body configured to engage a patient access point, the patient interface body including a distal end configured to facilitate insertion into the patient access point and a proximal end including a lip defining a cuff therein, the patient interface body including a channel defined therethrough;
a flexible seal configured to securely seat within the cuff, the flexible seal including an instrument opening defined therein configured to align in registration with the channel;
a seal guard configured to sit atop the flexible seal, the seal guard including an instrument opening defined therein configured to align in registration with the channel and the flexible seal instrument opening; and
a port cover configured to securely engage the lip to encapsulate the flexible seal and the seal guard, the port cover defining an opening configured to align in registration with the channel, the flexible seal instrument opening and the seal guard opening.

2. The instrument port for surgical instruments according to claim 1, wherein the patient access point is a wound, mechanical instrument ring or natural orifice of a patient and the patient interface body is configured to mechanically engage the wound, mechanical instrument ring or natural orifice.

3. The instrument port for surgical instruments according to claim 1 wherein the flexible seal is made from silicone.

4. The instrument port for surgical instruments according to claim 1 wherein the seal guard is made from a material that is harder than the flexible seal.

5. The instrument port for surgical instruments according to claim 1 wherein the seal guard is made from a material that is more lubricous than the flexible seal.

6. The instrument port for surgical instruments according to claim 1 wherein the opening defined in the flexible seal is smaller than the opening defined in the port cover.

7. The instrument port for surgical instruments according to claim 1 wherein the opening defined in the seal guard is smaller than the opening defined in the flexible seal.

8. The instrument port for surgical instruments according to claim 1 wherein the flexible seal and the seal guard each include a series of additional openings defined therein configured to operably engage a corresponding plurality of bosses disposed within the cuff.

9. The instrument port for surgical instruments according to claim 1 wherein the port cover is secured to the lip via screws that operably engage the lip to the port cover.

10. The instrument port for surgical instruments according to claim 8 wherein the port cover is secured to the lip via a plurality of screws that operably engage the corresponding plurality of bosses disposed within the cuff.

11. An instrument port for surgical instruments, comprising:
a patient interface body configured to engage a patient access point, the patient interface body including a distal end configured to facilitate insertion into the patient access point and a proximal end including a lip defining a cuff therein, the patient interface body including a channel defined therethrough extending from the proximal end to the distal end;
a flexible seal configured to securely seat within the cuff, the flexible seal including an instrument opening defined therein having a first diameter and configured to align in concentric registration with the channel;
a seal guard configured to sit atop the flexible seal, the seal guard including an instrument opening defined therein having a second diameter smaller than the first diameter and configured to align in concentric registration with the channel and the flexible seal instrument opening; and
a port cover configured to securely engage the lip to encapsulate the flexible seal and the seal guard, the port cover defining an opening having a third diameter slightly larger than the first diameter and configured to align in concentric registration with the channel, the flexible seal instrument opening and the seal guard opening.

12. The instrument port for surgical instruments according to claim 11, wherein the patient access point is a wound, mechanical instrument ring or natural orifice of a patient and the patient interface body is configured to mechanically engage the wound, mechanical instrument ring or natural orifice.

13. The instrument port for surgical instruments according to claim 11 wherein the flexible seal is made from silicone.

14. The instrument port for surgical instruments according to claim 11 wherein the seal guard is made from a material that is harder than the flexible seal.

15. The instrument port for surgical instruments according to claim 11 wherein the seal guard is made from a material that is more lubricous than the flexible seal.

16. The instrument port for surgical instruments according to claim 11 wherein the flexible seal and the seal guard each include a series of additional openings defined therein configured to operably engage a corresponding plurality of bosses disposed within the cuff.

17. The instrument port for surgical instruments according to claim 11 wherein the port cover is secured to the lip via screws that operably engage the lip to the port cover.

18. The instrument port for surgical instruments according to claim 16 wherein the port cover is secured to the lip via a plurality of screws that operably engage the corresponding plurality of bosses disposed within the cuff.

19. The instrument port for surgical instruments according to claim 11 wherein the patient interface body includes helical threads to facilitate insertion of the instrument port within the patient access point.
